(19) United States
(12) Patent Application Publication
(10) Pub. No.: US 2012/0024974 A1
(43) Pub. Date: Feb. 2, 2012

(54) CUSTOMIZED DESIGNED FRAGRANCE SYSTEM

(75) Inventors: Mark Grodsky, Phoenix, AZ (US); Richard Neill, Atlanta, GA (US); Alicia Thompson, Peoria, AZ (US)

(73) Assignee: Rich Brands LLC, Phoenix, AZ (US)

(21) Appl. No.: 12/846,559

(22) Filed: Jul. 29, 2010

(51) Int. Cl. A61L 9/04

(52) U.S. Cl. 239/6; 239/34; 239/44

(57) ABSTRACT

A fragrance dispenser in constructed of a dispenser body having a top end, a bottom end, a front, a back, a left side and a right side. The front side includes a window to permit at least a portion of the interior of the dispenser body to be viewed from without. Also, the top end includes at least one opening for emitting a fragrance. A container is disposed within the dispenser body and is adapted to hold a liquid fragrance that is to be dispensed. This container is viewable through the window. Further, a dispensing mechanism is disposed within the dispenser body and is configured to dispense liquid fragrance from the container into the atmosphere.

### BACKGROUND OF THE INVENTION

This invention relates generally to the field of fragrance dispensers, and in particular to customizable fragrance dispensers.

A wide variety of fragrance dispensers and air freshening devices are currently being marketed. For example, air fresheners having a given fragrance can be purchased at a wide variety of retail locations. Currently, there are few options that permit a customer to customize a fragrance dispenser, both in terms of a given fragrance as well as the appearance of the fragrance dispenser.

### BRIEF SUMMARY OF THE INVENTION

The invention provides fragrance dispensers that can be customized to accommodate the tastes of consumers. In so doing, the fragrance dispensers themselves include features that make them amenable customization. Additionally, various methods are provided to permit fragrance dispensers to be customized, both in terms of their aesthetics as well as the fragrances that they dispense.

In one exemplary embodiment, a fragrance dispenser comprises a dispenser body having a top end, a bottom end, a front, a back, a left side and a right side. The front side includes a window to permit at least a portion of the interior of the dispenser body to be viewed from without. Also, the top end includes at least one opening for emitting a fragrance. A container is disposed within the dispenser body and is adapted to hold a liquid fragrance that is to be dispensed. This container is viewable through the window. Further, a dispensing mechanism is disposed within the dispenser body and is configured to dispense liquid fragrance from the container into the atmosphere.

In one aspect, the dispenser body has a central vertical axis running from the bottom side to the top side, midway between the left side and the right side. Also, the front side tapers inward toward the central axis. In another aspect, the dispenser body has a top portion and a bottom portion that are separable from each other. The container is deposited in the bottom portion. In a further aspect, the window is rectangular in geometry and is disposed in the front side of the bottom portion to permit visualization of the container. In this way, the color of the fragrance can be viewed through the window.

One way to customize the dispenser is through the use of one or more skins that are positioned over or about the dispenser body and/or the container. For example, in one embodiment, a top skin is configured to be adhered to the top portion of the dispenser body. The top skin includes at least one color and at least one design. For instance, the design could be the logo of a sports team, university, company or the like. As another example, a bottom skin is configured to be adhered to the bottom portion of the dispenser body. The bottom skin is sized so as to not interfere with the widow when positioned over the bottom portion of the dispenser body. For instance, the skin may contain an opening that is aligned with the window when adhered to the bottom portion. As still another example, a container skin may be placed onto the container to change the visual appearance of the container when viewed through the window. For instance, the color of the container skin may be selected based on the type of fragrance, e.g., pink for a rose scent.

In one arrangement, the container includes a wick, and the dispensing mechanism comprises a ceramic heater that is positioned to heat the wick and diffuse the fragrance into the atmosphere. An electrical plug may be electrically coupled to the ceramic heater. In a further aspect, at least one button may be provided to release an interlock between the top portion and the bottom portion.

The dispensing unit may be sold alone, or as part of a system or kit. Such a system may include the dispensing unit, one or more skins, and/or one or more fragrances.

A variety of methods may be employed to customize a fragrance dispenser. This could be done at the retail level or on-line. For example, at the retail level, the dispensing unit, fragrance and/or skins may be sold separately to allow the consumer to mix and match. These same components may be purchased on-line, such as by using a web site. In one on-line method, a first data file is transmitted from a computer system to a user computer. The first data file comprises data for displaying with the user computer a dispensing unit and examples of skins that may be placed about the dispensing unit. A second data file may be transmitted from the computer system to the user computer that comprises data for displaying with the user computer options for customizing the cover. The options may include an image to be included on the skin and/or a color for the skin. The computer system receives an order from the consumer that includes a selection of a skin with at least one of the options for customizing the skin. The computer system then transmits instructions to fulfill the order.

The options for skins may include a top skin for the top portion of the dispenser, a bottom skin for the bottom portion of the dispenser, and/or a container skin for covering the container. In addition, the consumer may also order a dispensing unit and/or one or more fragrances.

### BRIEF DESCRIPTION OF THE DRAWINGS

FIG. 1 is a front perspective view of one embodiment of a fragrance dispenser according to the invention.

FIG. 2 is a rear perspective view of the fragrance dispenser of FIG. 1.

FIG. 3 is a bottom view of the fragrance dispenser of FIG. 1.

FIG. 4 is a right side view of the fragrance dispenser of FIG. 1.

FIG. 5 is a top view of the fragrance dispenser of FIG. 1.

FIG. 6 is an assembly view of the fragrance dispenser of FIG. 1.

FIG. 7 illustrates one embodiment of a fragrance dispensing kit according to the invention.

FIG. 8 illustrates one method for disassembling a fragrance dispenser of the kit of FIG. 7.

FIG. 9 illustrates a method for applying a skin to a top portion of the fragrance dispenser of the kit of FIG. 7.

FIG. 10 illustrates a further step of applying the skin of FIG. 9.

FIG. 11 illustrates still a further step of applying the skin of FIG. 9.

FIG. 12 illustrates a step of applying a skin to a bottom portion of the fragrance dispenser of FIG. 7.

FIG. 13 illustrates a step of applying a skin to a fragrance container of the kit of FIG. 7.

FIG. 14 illustrates a further step of applying the skin to the fragrance container of FIG. 13.

FIG. 15 illustrates a step of unscrewing a cap of the fragrance container of the kit of FIG. 7.

FIG. 16 illustrates a step of inserting the fragrance container into the bottom portion of the fragrance dispenser of FIG. 7.

FIG. 17 illustrates the step of connecting the bottom portion of the fragrance dispenser back together with the top portion.

FIG. 18 illustrates the step of plugging the fragrance dispenser of FIG. 7 into an electrical outlet.

FIG. 19 is a schematic system diagram describing a system that may be employed to order a fragrance kit or components for constructing a fragrance kit according to the invention.

FIG. 20 is a schematic diagram of a computer system that may be employed to order various components of a fragrance kit according to the invention.

### DETAILED DESCRIPTION OF THE INVENTION

The invention provides fragrance dispensers, fragrances, covers for fragrance dispensers and methods for customizing fragrance dispensers. The fragrance dispensers are constructed of a dispenser body that may have one or more parts. For example, the fragrance dispensers may have a top part and a bottom part that are separable from each other in order to provide access into the interior of the dispenser so that the fragrance can be inserted. The fragrance body may be provided in a neutral color, such as white, so that the covers may be used to decorate the dispenser. Similarly, the containers holding the fragrances can have a neutral color, or be clear, so that these too can be customized using a skin. In this way, a consumer may purchase a dispensing unit, then have options for selecting different fragrances, as well as decorative skins for the dispensing unit and/or the container holding the fragrance.

To facilitate customization, a variety of marketing channels may be used. For example, some or all of the components (dispensing unit, covers and fragrances) could be sold at the retail level (brick and mortar stores or mail order catalogs) and/or on on-line using a web site. The components may be all sold separately or sold together as kits. For instance, a kit could include a dispensing unit and one or more fragrances. Or, the kit could also include one or more skins. Skins and/or replacement fragrances could also be purchased separately.

Further, the separate components or kits could be sold through the same or different marketing channels. As one example, a consumer may purchase a dispensing unit in a retail store or on-line. The consumer may also purchase a fragrance for the dispensing unit from the same channel or a different channel. This may be done at the same time, or a later time. For example, the consumer could purchase a dispensing unit at a retail location, then go home and purchase one or more fragrances on-line. In a similar manner, the skin(s) for the dispensing unit could be purchased along with the dispensing unit or be purchased at a separate time. For instance, if a consumer has a dispensing unit and wants to change the look of the dispensing unit, the consumer could go to a store or visit a web site to purchase a different skin(s). As an example, if the consumer's favorite sports team won a championship, a skin with the sports team's logo could be ordered and placed about the dispenser. Later, a different skin could be added to change the look of the dispenser. In a similar manner, the skin for the container that holds the fragrance could be changed, perhaps to suggest the type of fragrance being dispensed. For instance, lavender color could be used for a lavender scent. Or, it could simply be some whimsical pattern, or contain a logo. If another fragrance is used, a different skin for the fragrance container could be used.

The invention may utilize a variety of skins that may be adhered or attached to various components of the dispenser or fragrance container. The skins may comprise a flexible material, such as a vinyl, plastic, paper label, foils, or the like. Merely by way of example, the skin may comprise some type of appliqué with adhesive or other backing, a sticker, a decal, a wrapping, or the like. One exemplary material is a vinyl or PET material. This vinyl material may include a backing that may be peeled off prior to application of the skin to the dispenser body. The vinyl typically will not include an adhesive but will naturally stick to plastic or glass surfaces. However, in some cases an adhesive could be used so that the skin is permanently attached rather than removable. Such an adhesive is preferably a non-permanent glue or adhesive.

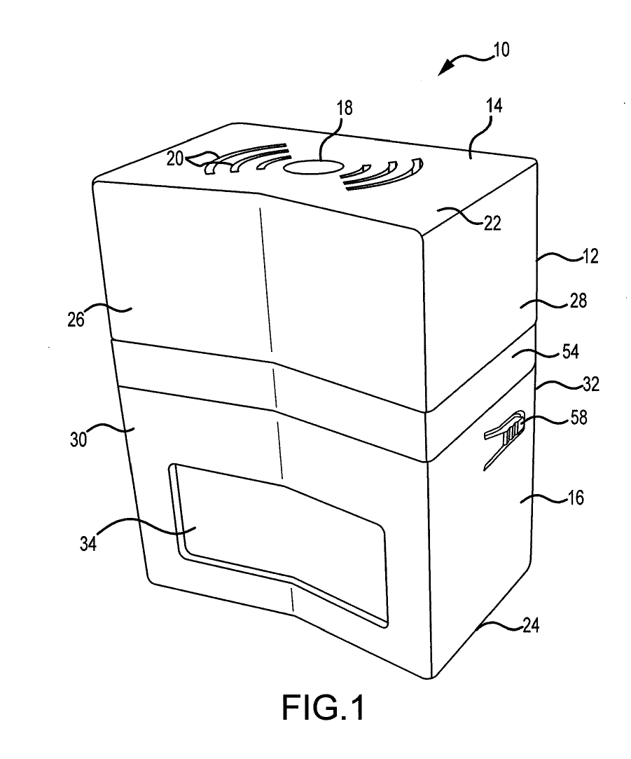

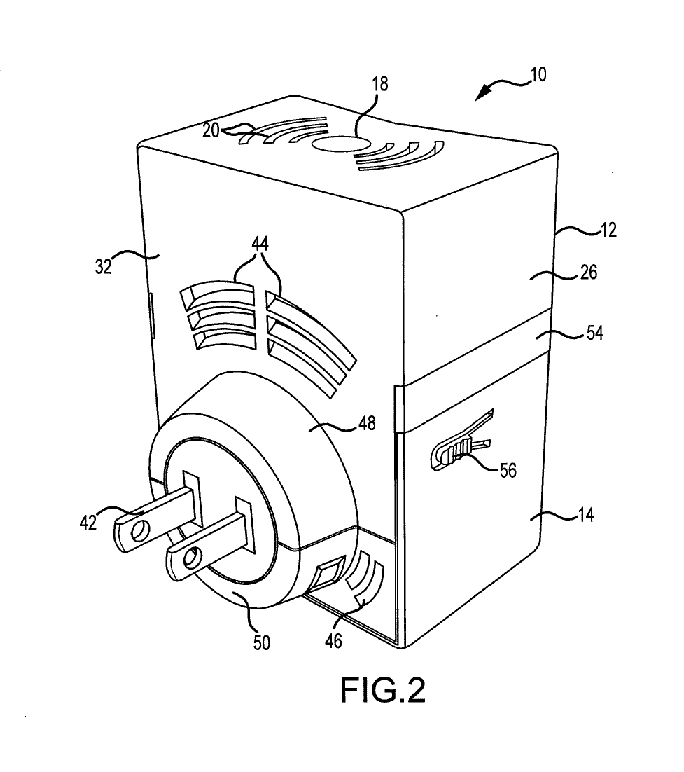

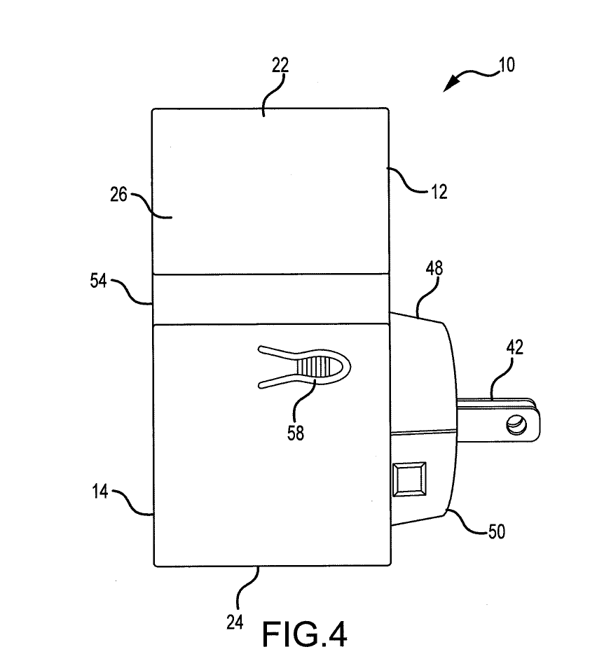

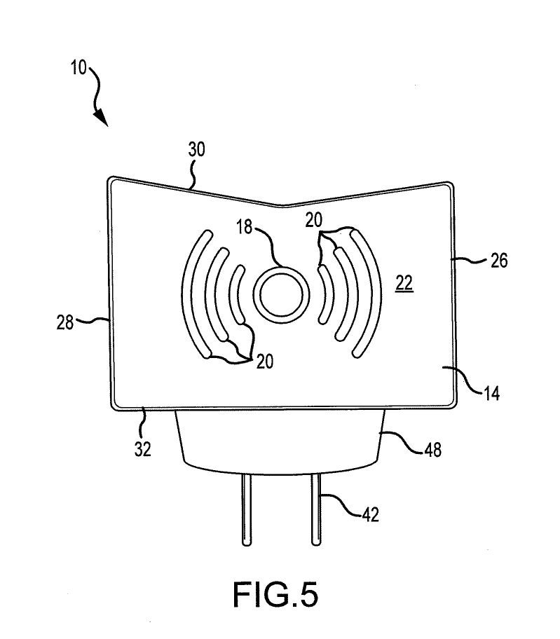

Referring now to FIGS. 1-5, one embodiment of a fragrance dispenser 10 will be described. Fragrance dispenser 10 comprises a dispenser body 12 having a top portion 14 that is separable from a bottom portion 16. As also shown in FIG. 5, top portion 14 has a central opening 18 and multiple vents 20. Central opening 18 and vents 20 are employed to emit a fragrance that is emitted from dispenser 10 as described hereinafter.

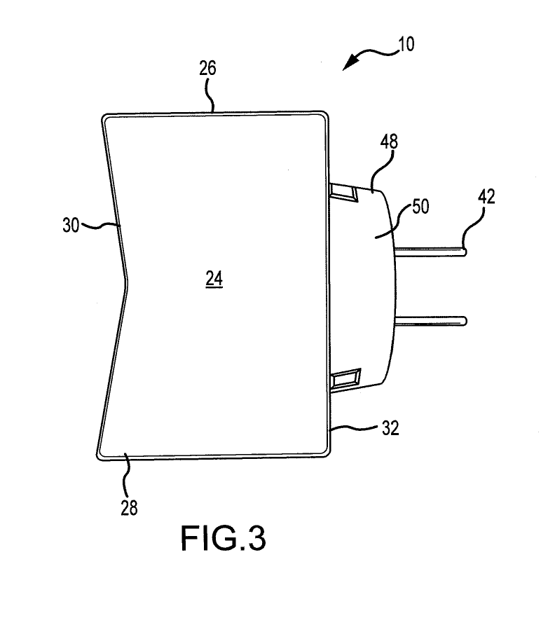

For convenience of discussion, dispenser body 12 may be defined in terms of a top end 22, a bottom end 24, a left side 26 and a right side 28. Also, dispenser body 12 may define a front 30 and a back 32. A central vertical axis extends from bottom end 24 to top end 22, generally through the area of central opening 18 as illustrated in FIG. 5. In other words, the central vertical axis is midway between left side 26 and right side 28. Further, front 30 tapers inward to the central axis from both the left side 26 and the right side 28 as best illustrated in the top and bottom views of FIGS. 3 and 5. The tapering of dispenser body 12 not only serves to provide the dispenser with an aesthetically pleasing appearance, but also is employed to accentuate the depiction of the color which is displayed through a window 34 in front 30 of bottom portion 16 as best illustrated in FIG. 1. By way of example, the distance from left side 26 to right side 28 may be in the range from about 4 cm to about 8 cm, and the distance from front 30 to back 32 along the outer edges may be in the range from about 3 cm to about 6 cm. In contrast, the distance from front 30 to back 32 at the central axis may be about 2 cm to about 5 cm, making the depth of the V-shaped notch up to about 1 cm, and in some cases about 0.5 cm. Also, in some cases, the front surface may be flat or planar. Typically, the distance from bottom end 24 to top end 22 will be in the range from about 6 cm to about 10 cm. These dimensions provide sufficient space to hold the fragrance and heating elements while also permitting it to be conveniently plugged into an electrical outlet as described hereinafter.

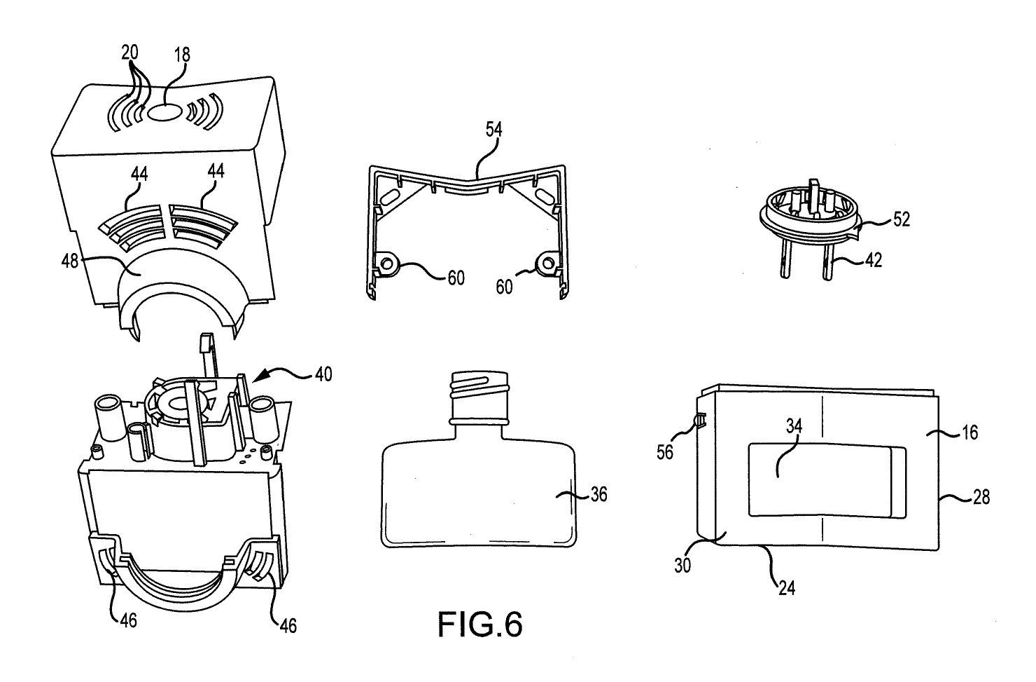

Referring also now to FIG. 6, construction of fragrance dispenser 10 will be described in greater detail. As shown, top portion 14 is separable from bottom portion 16. This is so that the internal components of fragrance dispenser 10 may be accessed. In particular, separation of top portion 14 from bottom portion 16 permits access into the interior space of the fragrance dispenser which holds a container 36 which in turn holds a fragrance that is to be dispensed. Conveniently, container 36 is sized and shaped so that it will fit within bottom portion 16. Further, a major surface of container 36 is visible through window 34 in bottom portion 16. In this way, the user is able to both see the volume of fragrance remaining within container 36 as well as to view a particular color of the fragrance and/or the container 36 as described hereinafter.

Fragrance dispenser 10 further includes a dispensing mechanism 38 which comprises a ceramic heater 40. In turn, ceramic heater 40 receives power from an electric plug 42 which is sized and configured to be inserted into a conventional 110V outlet. Alternatively, plug 42 could be configured for other voltages such as those used in many international countries. When plug 42 is inserted into an electric outlet, electrical power is transferred via wires (not shown) to a ceramic heating element in ceramic heater 40. A wick (not shown) extends vertically upward from container 36 and is surrounded by the annular ceramic element. As the annular ceramic element is heated, it accelerates vaporization of the fragrance contained within 36 which passes upwardly through central opening 18 and vents 20 of top portion 14. Central opening 18 and vents 20 are designed to maximize the amount of fragrance that is dispensed from fragrance dispenser 10 while still being able to provide the tapered outer features as previously described. Top portion 14 also includes back vents 44 which provide adequate ventilation to fragrance dispenser 10 and enhance airflow upwardly through dispenser body 12 so that the vaporized fragrance may more easily be dispensed from central opening 18 and vents 20. Dispensing mechanism 38 may also include vents 46 to facilitate this process.

Top portion 14 includes a semi circular region 48, and dispensing mechanism 38 also includes a semi circular region 50 that cooperate together to hold plug 42 in place once fragrance dispenser 10 is assembled. Semi circular regions 48 and 50 may include a groove for receiving a circular protrusion 52 on plug 42.

An insert 54 may be coupled to top portion 14 to facilitate coupling of top portion 14 to bottom portion 16 when fragrance dispenser 10 is fully assembled. More specifically, bottom portion 16 includes a left tab 56 and a right tab 58 that interact with holes 60 in insert 54. Once top portion 14 and bottom portion 16 are slid adjacent each other, left tab 56 and right tab 58 snap into holes 60. When ready to remove top portion 14, tabs 56 and 58 are depressed to remove them from holes 60. In this way, top portion 14 may be slid from bottom portion 16 to access the interior of fragrance dispenser 10.

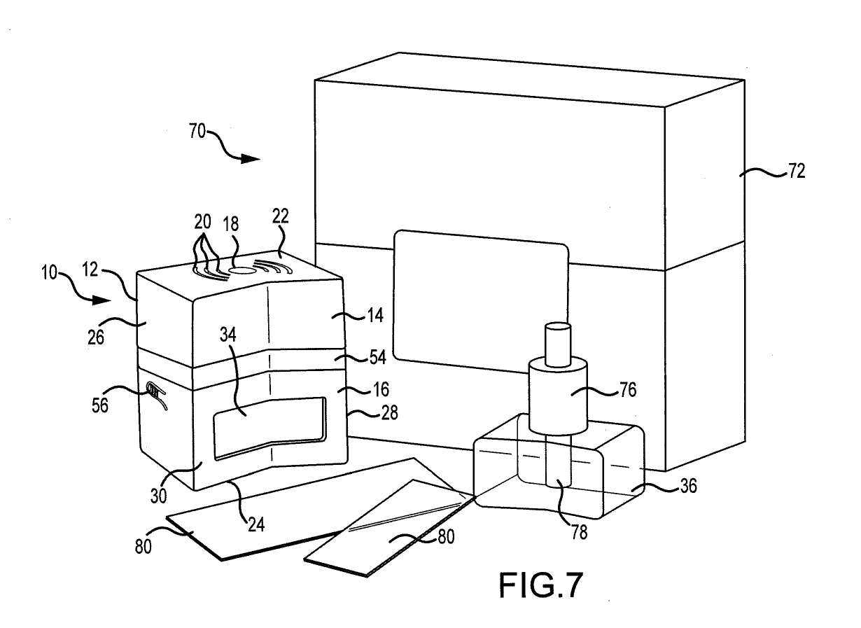

Referring now to FIGS. 7-18, one exemplary method for utilizing fragrance dispenser 10 will be described. In one embodiment, fragrance dispenser 10 may be included as part of a kit 70 that includes a package 72 for containing all of the components. Package 72 may comprise a box or other container that is sized to hold fragrance dispenser 10 and one or more containers 36 which hold a fragrance that is to be dispensed. Package 72 may conveniently include a window 74 for viewing the components of kit 70. When included within kit 70, container 36 will include a lid 76 that may be screwed or snapped to container 36 so that the liquid fragrance within container 36 will not spill or evaporate. Also, a wick 78 extends vertically upward through container 36 and is enclosed by lid 76 to prevent evaporation of the liquid fragrance.

Kit 70 also includes multiple skins that may be attached to fragrance dispenser 10 and/or container 36. These skins are generally designated with reference numeral 80. As described hereinafter, skins 80 permit fragrance dispenser 10 and/or container 36 to be customized to include various colors, logos, language, designs, or the like. For example, each dispensing kit 70 could include multiple types of skins so that the user may mix and match so as to be able to customize the fragrance dispenser. Also, skins 80 and/or container 36 may be sold separately so that even if a customer purchases a kit, the customer is provided with the option of redesigning the overall appearance by selecting different skins and/or fragrances.

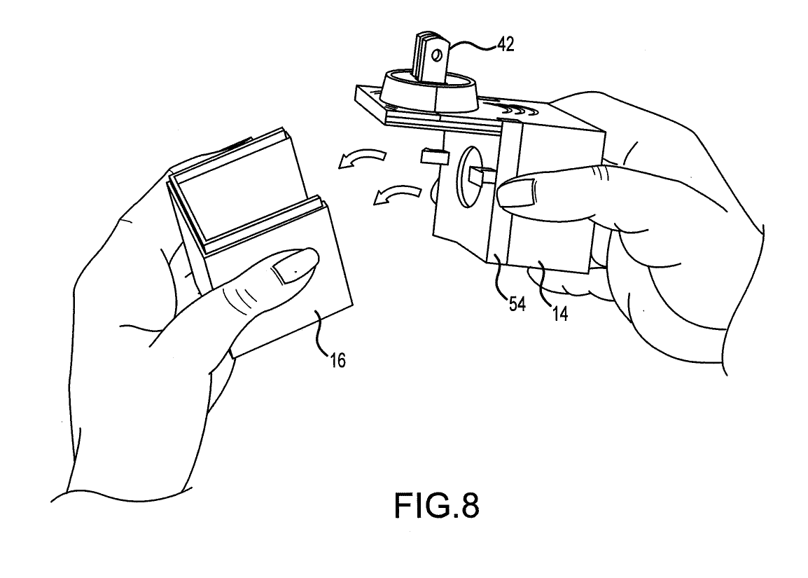

As shown in FIG. 8, to begin the customization process top portion 14 may be separated from bottom portion 16. This is accomplished by depressing left tab 56 and right tab 56 and sliding top portion 14 from bottom portion 16.

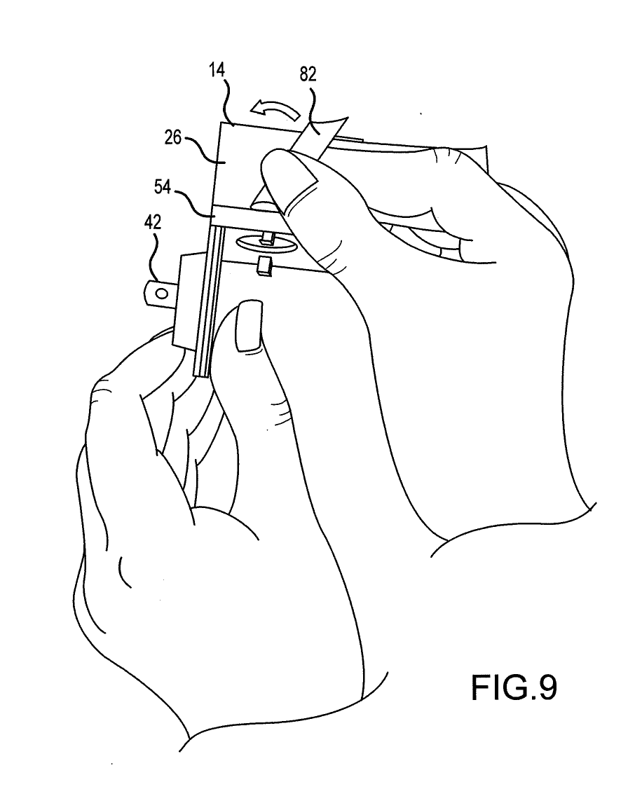

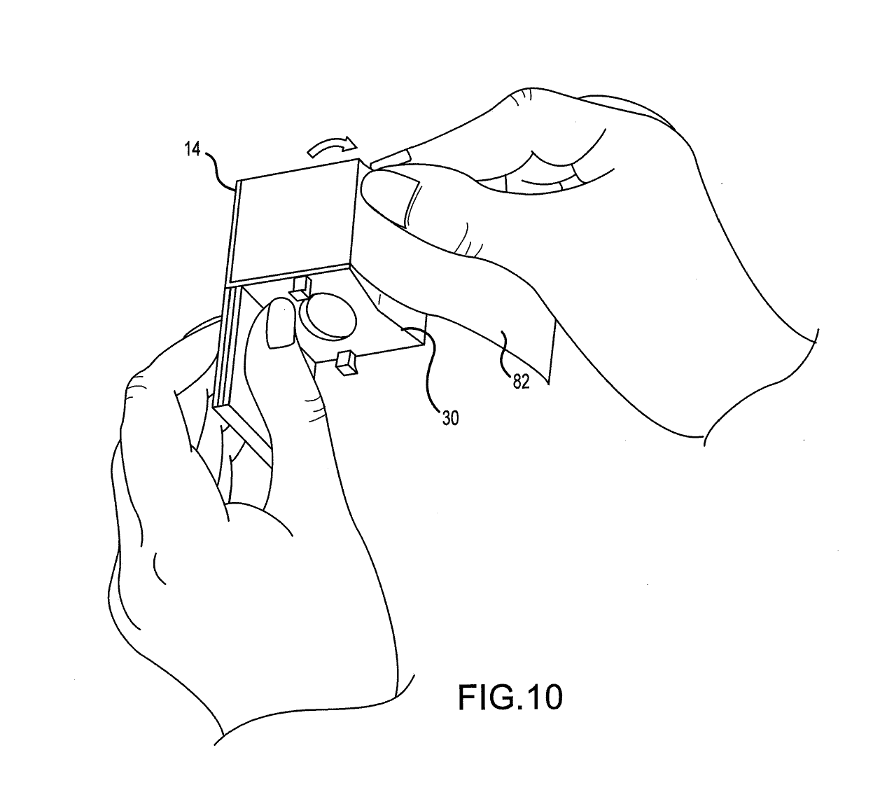

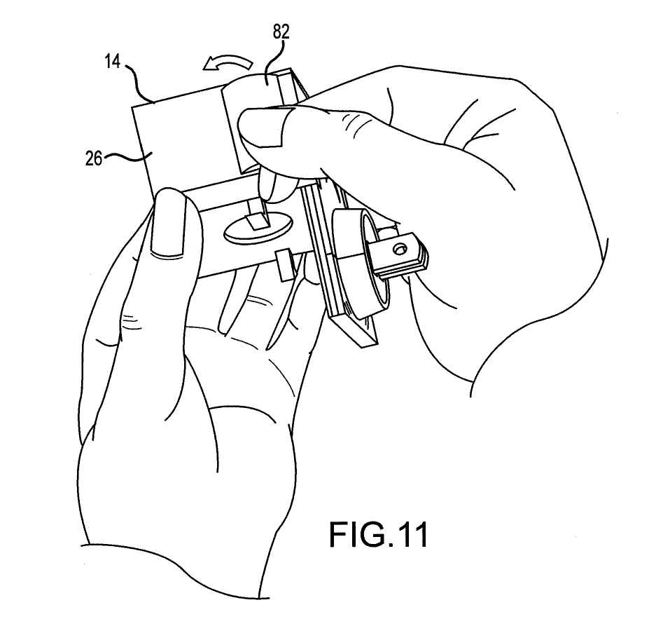

The user then selects a skin 82 for top portion 14 and removes the backing from skin 82. As shown in FIG. 9, the user may begin from left side 26 of top portion 14 and begin applying the skin, slowly adhering it to the outer surface. As shown in FIG. 10, once skin 82 is attached to left side 26 it is wrapped about front 30 of top portion 14 and then wrapped around to left side 26 as shown in FIG. 11. Skin 82 is sized so that it generally covers the bulk of the surfaces of the left side, front and right side of top portion 14.

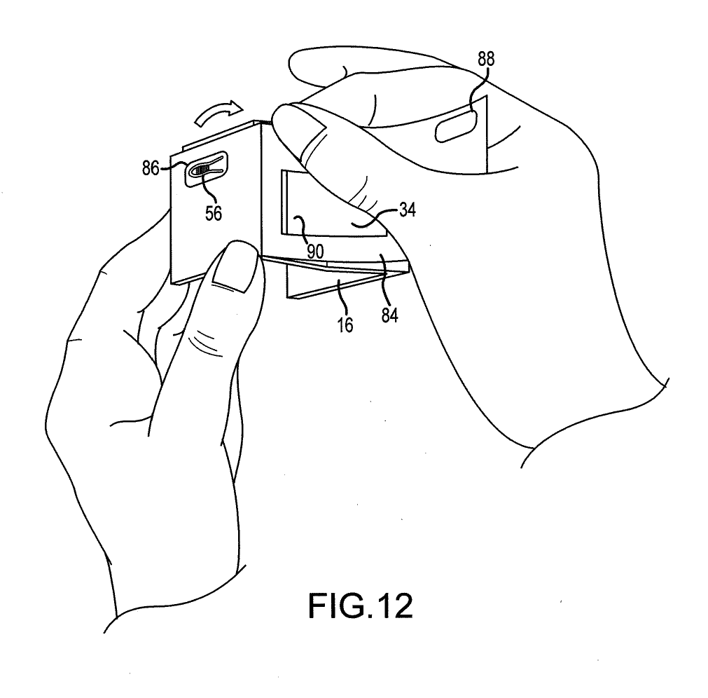

This same process may generally be repeated for bottom portion 16. More specifically, as shown in FIG. 12 the user selects a skin 84 for bottom portion 16. Conveniently, skin 84 may include a left opening 86, a right opening 88 and a central opening 90 for receiving left tab 56, right tab 58 and window 34, respectively. Once skin 84 is selected, the backing is removed and beginning with the left side, skin 84 is placed onto the surface of bottom portion 16 so that left opening 86 is aligned with left tab 56. Then, skin 84 is adhered to the front of bottom portion 16 with central opening 90 aligned with window 34. Finally, skin 86 is placed about the right side, with right opening 88 positioned about the right tab 58.

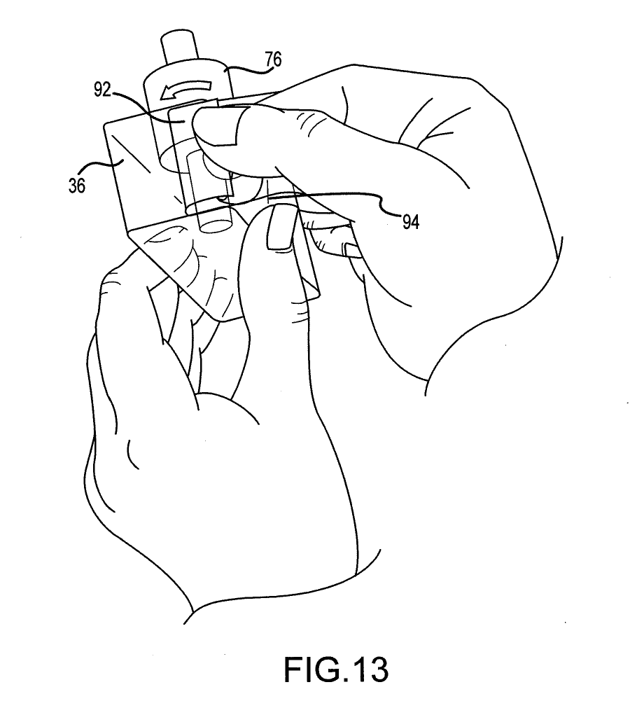

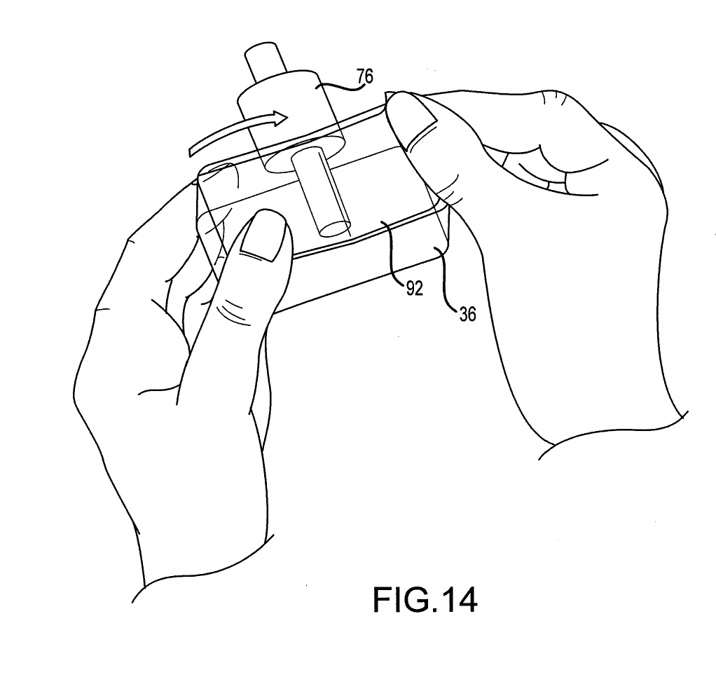

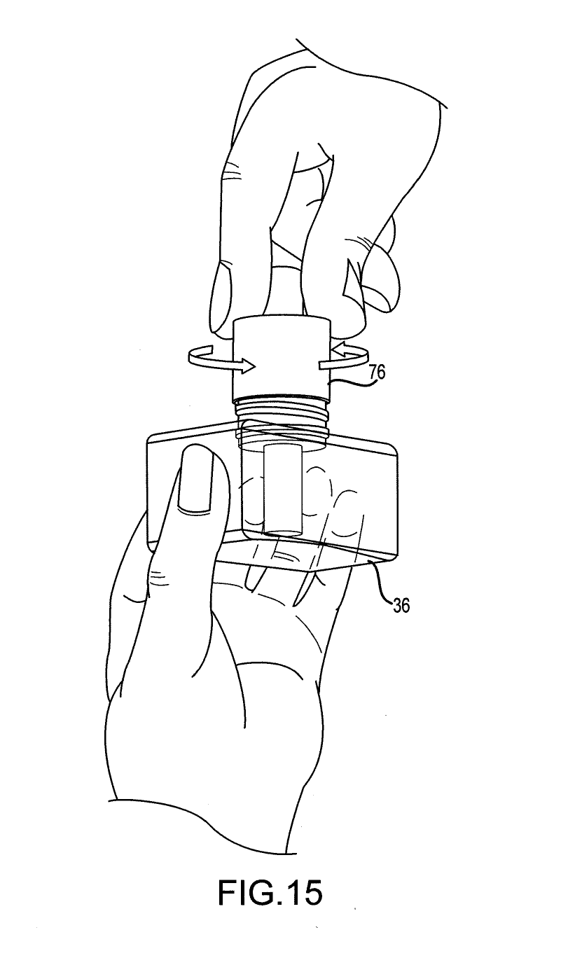

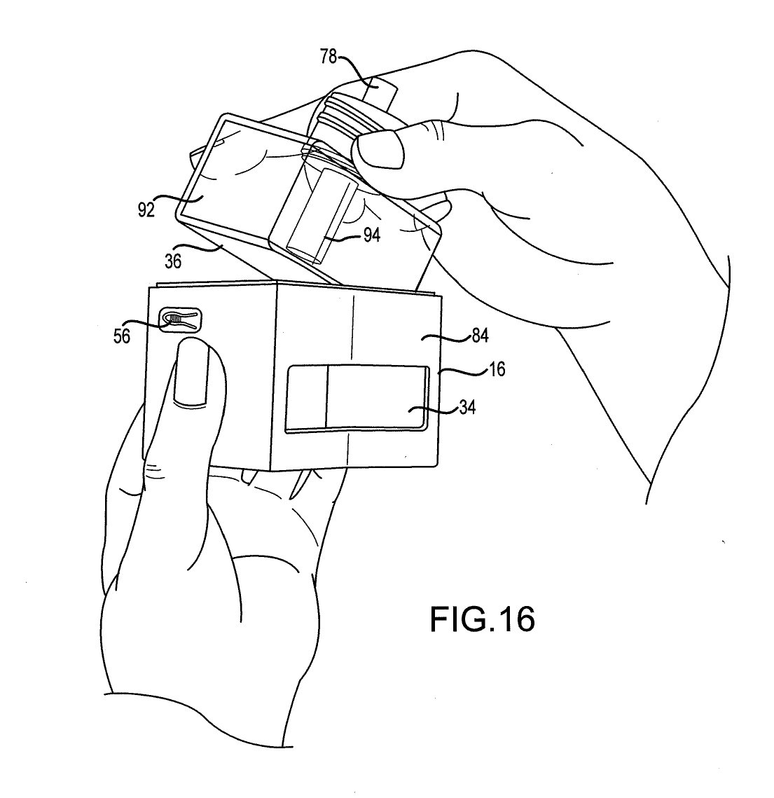

As illustrated in FIG. 13, container 36 may also be customized by selecting a skin 92 which preferably has a certain color and/or design. For example, skin 92 may be a transparent colored skin that allows the user to see the contents of container 36 while altering the color of its contents. To attach skin 92, the user removes the protective backing and then wraps it about the container 36 as illustrated in FIG. 13. The front side of container 36 includes an indent 94 such that container 36 tapers inward toward indent 94 in a manner similar to dispenser 10. As further illustrated in FIG. 14, skin 92 is placed completely over the front of container 36 so that as it is viewed through window 34 it will have the color of the skin 92. Once fragrance dispenser 10 and container 36 have been customized using their respective skins, the dispenser 10 is ready to be put back together. As illustrated in FIG. 15, lid 76 is unscrewed from container 36, thereby revealing the protruding wick. With lid 36 removed, container 36 is inserted into bottom portion 16 as illustrated in FIG. 16. As previously described, the front surface of container 36 is tapered to match the tapering of the front of bottom portion 16. In this way, container 36 will fit within bottom portion 16 in only one orientation. This helps to insure that the skin is aligned with the window.

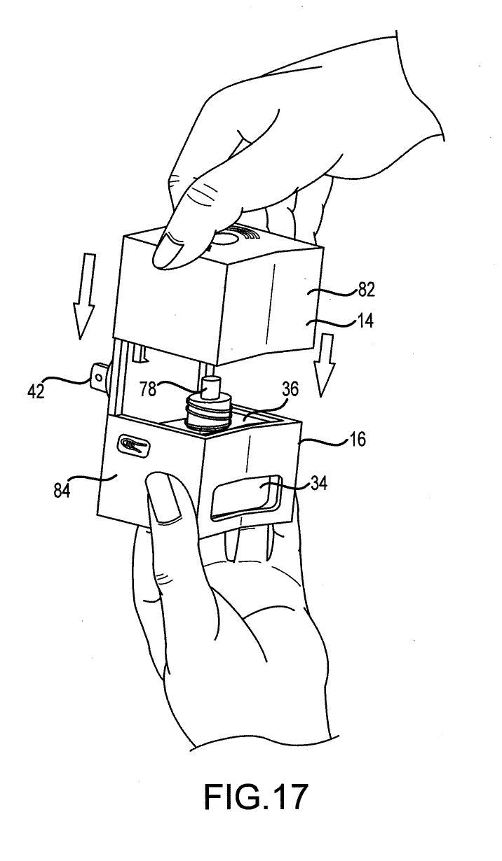

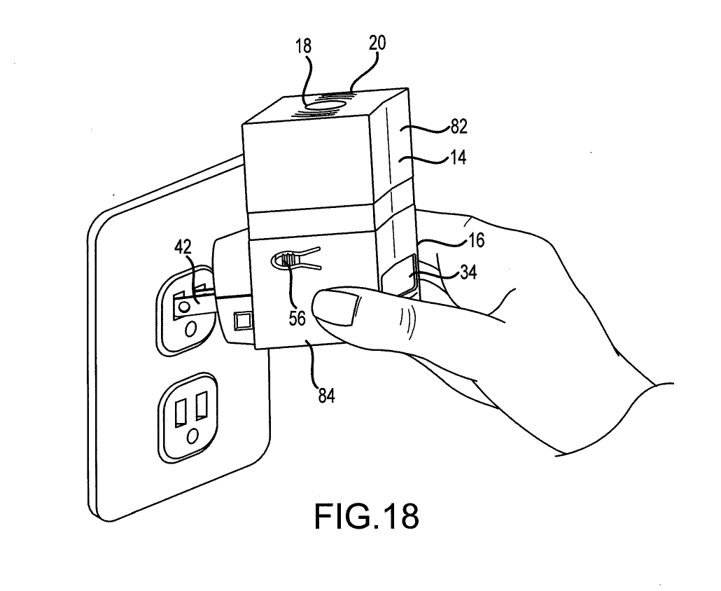

As illustrated in FIG. 17, top portion 14 is then slid onto bottom portion 16 until it is snapped in place via tabs 56 and 58. Further, dispenser 10 is now customized and ready to be operated. This is accomplished by inserting plug 42 into an electrical outlet as illustrated in FIG. 18. Once within the wall, the user is able to visualize skins 82 and 84 that were placed onto the outer surface of dispenser body 12. Further, because skin 92 was placed about container 36, the liquid fragrance within container 36 will have a color based on the color selected for skin 92.

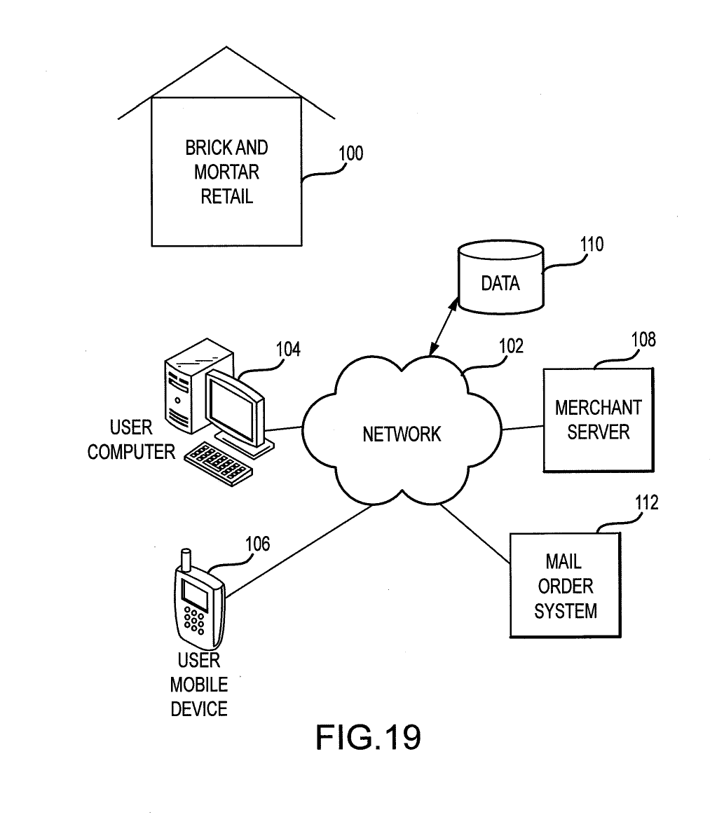

FIG. 19 illustrates the various ways in which the components of a fragrance dispenser kit can be purchased. For example, fragrance dispenser kits of the type described herein may be purchased at a brick and mortar retail store 100. The kits may include a fragrance dispenser along with one or more skins and one or more fragrance containers. In some cases, the components could be sold separately so that a customer can purchase only skins to modify the décor of an existing fragrance dispenser. Similarly, fragrances and skins for those fragrances could also be purchased separately.

In a similar manner, kits or various components of the kits (including the skins), may be purchased over a network 102. The network 102 may be any type of network familiar to one skilled in the art that can support data communications using any of a variety of commercially available protocols, such as TCP/IP, SNA, IPX, AppleTalk, and the like. As one example, network 102 may be a local area network (“LAN”) such as an Ethernet network, a token ring network, a wide area network, a virtual network, a virtual private network, the Internet, and intranet and extranet, a public switched telephone network (“PSTN”), in infrared network, a wireless network, including networks operating under the IEE802.11 suite of protocols, the Blue Tooth protocol, or other wireless protocol, or other types of networks including GSM, 3G, 4G, and the like. To order any of the components of a kit a personal computer 104 may be employed. Computer 104 may be any type of computer known in the art, including personal computers and/or laptop computers running various operating systems, such as Microsoft Corporation's Windows or Apple Corporation's Macintosh or Leopard Operating Systems. Alternatively, a user mobile device 106 may be used to view and order various components of the fragrance kit. Such a user mobile device may have both voice and data transfer capabilities, including those currently being marketed by BlackBerry, Apple's iPhone, and the like.

Typically, the user will use a computing device to access a merchant server 108 that is able to render an image on the display screen over the World Wide Web. The server 108 may be any type of general purpose computer and/or specialized server computer, such as PC servers, UNIX servers, mid range servers, main frame computers, rack mounted servers, and the like. The website accessed by the user may have various displays that permit the user to customize a particular skin. For example, the website may list various sports teams, countries, decorating genres, and the like. By clicking on any of these links, the user may be directed to images showing skins with certain sports logos, colors, designs, and the like. In some cases, the user could upload a photo or design of their own so that the skin may include a particular feature from the user. In a similar manner, the user may select various fragrances. After making the selection, these items may be placed into a checkout cart as is known in the art. Upon receipt of payment information, the purchased components may be shipped to a location designated by the user. In order to produce the web page and/or to provide or facilitate payment and shipping, the system may also include a database of enabling data 110. By way of example, database 110 may reside on a storage medium local to or resident in one or more of the computers on the network 102 including merchant server computer 108. Alternatively, database 110 may be remote from all of the computers. Any necessary files for performing the functions on any of the computers on network 102 may be stored in database 110.

Also coupled to network 102 may be a mail order system 112 that may receive orders from a mail order catalogue. This catalogue may offer all of the components in a kit or may sell them separately, such as the fragrance dispenser, skins and/or the fragrance. These components may be ordered using computer 104 or user mobile device 106.

In summary, any of the kits described herein or their various components may be purchased in whole or in part in a separate brick and mortar location and/or online over a network. Further, the skins may be selected from an assortment already prepared skins that may be used to customize the look and feel of the fragrance dispenser or fragrance container. Another option is to permit the user to construct their own skins by uploading photos or other images or designs which can be manufactured into a skin and then provided to the consumer.

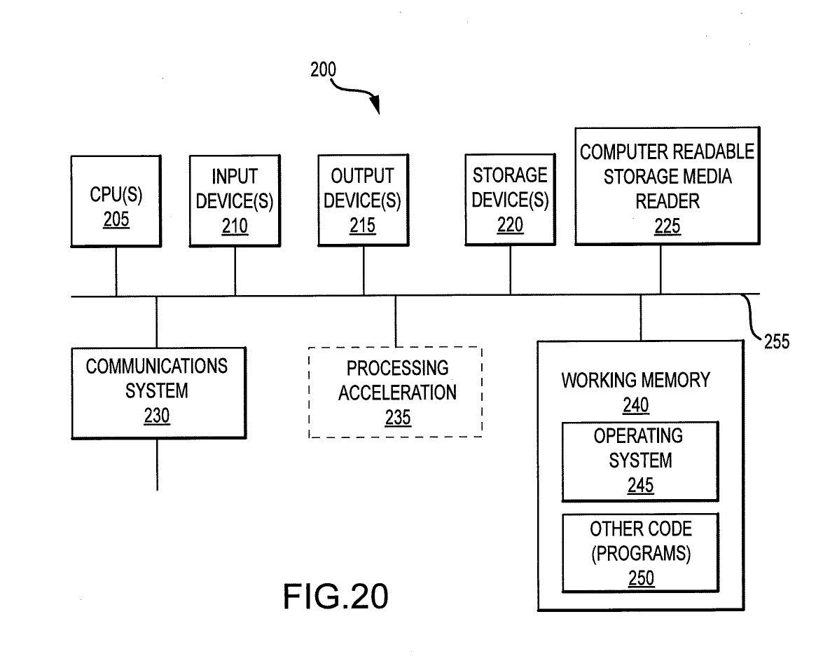

FIG. 20 illustrates a block diagram of an exemplary computer system upon which various elements of the environment illustrated in FIG. 19 may be implemented. The computer system 200 is shown comprising hardware elements that may be electively coupled by use of a bus 255. The various hardware elements may include one or more central processing units 205, one or more input devices 210 (such as a scanner device, a mouse, a keyboard), and one or more output devices 215, such as a display device, a printer, or the like. Computer system 200 may also include one or more storage devices 220. For instance, storage device 220 may be a disc drive, optical storage device, solid state storage device, such as a random access memory and/or a read only memory, which can be programmable, flash updatable and/or the like.

Computer system 200 may additionally include a computer readable storage media reader 225, a communication system 230 such as a modem, and working memory 240. In some embodiments, computer system 200 may also include a processing acceleration unit 235.

The computer readable storage media reader 225 can be connected to a computer readable storage medium which together may represent remote, local, fixed and/or removable storage devices. The communication system 230 may permit data to be exchanged with a network and/or any other computer or other type of device.

The computer system 200 may also comprise software elements that may be stored within a working memory 240, including an operating system 245 and/or other code 250, such as an application program. The application programs may implement the methods of the invention herein, including the rendering of various web pages when ordering or designing fragrance kits and/or skins.

The invention has now been described in detail for purposes of clarity and understanding. However, it will be appreciated that certain changes and modifications may be practiced within the scope of the appended claims.

What is claimed is:

1. A fragrance dispenser, comprising:
a dispenser body having a top end, a bottom end, a front, a back, a left side and a right side, wherein the front side includes a window to permit at least a portion of the interior of the dispenser body to be viewed from without, and wherein the top end includes at least one opening for emitting a fragrance;
a container disposed within the dispenser body, the container being adapted to hold a liquid fragrance that is to be dispensed, wherein the container is viewable through the window; and
a dispensing mechanism disposed within the dispenser body that is configured to dispense liquid fragrance from the container into the atmosphere.

2. A dispenser as in claim 1, wherein the dispenser body has a central vertical axis running from the bottom side to the top side, midway between the left side and the right side, and wherein the front side tapers inward toward the central axis.

3. A dispenser as in claim 1, wherein the dispenser body has a top portion and a bottom portion that are separable from each other, wherein the container is deposited in the bottom portion, and wherein the window is rectangular in geometry and is disposed in the front side of the bottom portion to permit visualization of the container.

4. A dispenser as in claim 1, wherein the container includes a wick, and wherein the dispensing mechanism comprises a ceramic heater that is positioned to heat the wick and diffuse the fragrance into the atmosphere.

5. A dispenser as in claim 3, further comprising a top skin that is configured to be adhered to the top portion of the dispenser body.

6. A dispenser as in claim 5, wherein the top skin includes at least one color and at least one design.

7. A dispenser as in claim 3, further comprising a bottom skin that is configured to be adhered to the bottom portion of the dispenser body.

8. A dispenser as in claim 7, wherein the bottom skin includes an opening that is aligned with the widow when positioned over the bottom portion of the dispenser body.

9. A dispenser as in claim 1, further comprising a container covering that is configured to be placed onto the container to change the visual appearance of the container when viewed through the window.

10. A dispenser as in claim 4, further comprising an electrical plug that is electrically coupled to the ceramic heater.

11. A dispenser as in claim 3, further comprising at least one button to release an interlock between the top portion and the bottom portion.

12. A method for customizing a fragrance dispenser, the method comprising:
transmitting a first data file from a computer system to a user computer, wherein the first data file comprises data for displaying with the user computer a dispensing unit and examples of skins that may be adhered to portions of the dispensing unit, wherein the dispensing unit comprises:
a dispenser body having a top end, a bottom end, a front, a back, a left side and a right side, wherein the front side includes a window to permit at least a portion of the interior of the dispenser body to be reviewed from without, and wherein the top end includes at least one opening for emitting a fragrance;
a container disposed within the dispenser body, the container being adapted to hold a liquid fragrance that is to be dispensed, wherein the container is viewable through the window; and
a dispensing mechanism disposed within the dispenser body that is configured to dispense liquid fragrance from the container into the atmosphere;
transmitting a second data file from the computer system to the user computer, wherein the second data file comprises data for displaying with the user computer options for customizing the skin, wherein the options include at least one of an image to be included on the skin and a color for the skin;
receiving at the computer system an order that includes a selection of a skin with at least one of the options for customizing the skin;
transmitting instructions from the computer system to fulfill the order.

13. A method as in claim 12, wherein the skin comprises a top skin, and wherein the order further includes a selection of a bottom skin.

14. A method as in claim 12, wherein the order further includes a selection of a container skin for covering the container.

15. A method as in claim 12, wherein the order further includes a dispensing unit.

16. A method as in claim 12, wherein the order further includes a fragrance.

17. A fragrance dispense system, comprising:
a fragrance dispenser, comprising a dispenser body having a top portion with a top end, a bottom portion having a bottom end, a front, a back, a left side and a right side, wherein the front side includes a window to permit at least a portion of the interior of the dispense body to be reviewed from without, and wherein the top end includes at least one opening for emitting a fragrance;
a container disposed within the dispenser body, the container being adapted to hold a liquid fragrance that is to be dispensed, wherein the container is viewable through the window; and
a dispensing mechanism disposed within the dispenser body that is configured to dispense liquid fragrance from the canister into the atmosphere.
a top portion skin that is configured to be adhered to the top portion of the dispenser body.

18. A system as in claim 17, further comprising a bottom portion skin that is configured to be adhered to the bottom portion of the dispenser body.

19. A system as in claim 17, further comprising a container skin that is configured to be placed onto the container to change the visual appearance of the container when viewed through the window.

20. A system as in claim 17, wherein the dispenser body has a central vertical axis running from the bottom side to the top side, midway between the left side and the right side, and wherein the front side tapers inward toward the central axis.
